(19) United States
(12) Patent Application Publication
(10) Pub. No.: US 2022/0004881 A1
(43) Pub. Date: Jan. 6, 2022

(54) CLINICAL MODEL GENERALIZATION

(71) Applicant: International Business Machines Corporation, Armonk, NY (US)

(72) Inventors: Sun Young Park, San Diego, CA (US); Dustin Michael Sargent, San Diego, CA (US)

(21) Appl. No.: 16/920,920

(22) Filed: Jul. 6, 2020

(51) Int. Cl. G06N 3/08; G16H 50/20; G06N 3/04

(52) U.S. Cl. CPC G06N 3/088; G06N 3/0454; G16H 50/20

(57) ABSTRACT

Provided is a method for adapting an artificial intelligence (AI) model. The method includes comparing a distribution of a clinical data characteristic of a genuine dataset with a target distribution of the clinical data characteristic to identify any categories of the clinical data characteristic that are underrepresented in the genuine dataset. The method further includes generating an artificial test dataset based on the result of the comparison. The method further includes generating training data based on the artificial test dataset. The method further includes providing the training data to the AI model to adapt the AI model.

### BACKGROUND

The present disclosure relates generally to the field of computer aided diagnosis (CAD) systems, and more particularly to the use of CAD in clinical model validation.

CAD systems are used in conjunction with artificial intelligence (AI) models to assist medical professionals in interpreting medical images. For example, CAD systems can be used to analyze digital images to identify patterns or anomalies. These identifications can then be used in clinical models to generate an indication of a potential issue or disease in the patient. This indication can be used to inform the medical professional's decision making processes.

### SUMMARY

Embodiments of the present disclosure include a method, computer program product, and system for adapting an artificial intelligence (AI) model. The method includes comparing a distribution of a clinical data characteristic of a genuine dataset with a target distribution of the clinical data characteristic to identify any categories of the clinical data characteristic that are underrepresented in the genuine dataset. The method further includes generating an artificial test dataset based on the result of the comparison. The method further includes generating training data based on the artificial test dataset. The method further includes providing the training data to the AI model to adapt the AI model.

Further embodiments of the present disclosure include a method, computer program product, and system for adapting an artificial intelligence (AI) model. The method includes comparing a distribution of a clinical data characteristic of a genuine dataset with a target distribution of the clinical data characteristic to identify an underrepresented category of the clinical data characteristic. The method further includes generating artificial data in the underrepresented category in the genuine dataset. The method further includes categorizing the artificial data. The method further includes applying a first discriminator to the artificial data to select artificial data that is categorized in the underrepresented category. The method further includes applying a second discriminator to the artificial data to remove artificial data that is categorized in a second category of the clinical data characteristic. The second category of the clinical data characteristic is another category of the clinical data characteristic that is represented in the genuine dataset, and the underrepresented category is different than the second category. The method further includes performing a statistical analysis of the artificial data to identify any problems with the artificial data. The method further includes generating training data based on the identified problems. The method further includes providing the training data to the AI model to adapt the AI model.

Further embodiments of the present disclosure include a method, computer program product, and system for clinical model generalization. The method includes analyzing at least one clinical data characteristic of a sample dataset. The method includes identifying a category of the at least one clinical data characteristic in which there is a discrepancy between an analyzed statistical distribution of data in the identified category and a target statistical distribution of data in the identified category. The method further includes generating synthetic data in the identified category based on the target statistical distribution of data in the identified category. The method further includes performing a performance analysis of the synthetic data to identify a problem with the synthetic data. The method further includes generating training data to address the identified problem.

The above summary is not intended to describe each illustrated embodiment or every implementation of the present disclosure.

### BRIEF DESCRIPTION OF THE DRAWINGS

The drawings included in the present disclosure are incorporated into, and form part of, the specification. They illustrate embodiments of the present disclosure and, along with the description, serve to explain the principles of the disclosure. The drawings are only illustrative of typical embodiments and do not limit the disclosure.

FIG. 1 illustrates a flowchart of an example method for adapting an AI model, in accordance with embodiments of the present disclosure.

FIG. 2 illustrates an example data table that can be used in the example method of FIG. 1, in accordance with embodiments of the present disclosure.

FIG. 3 illustrates a flowchart of an example method for generating artificial test data in the example method of FIG. 1, in accordance with embodiments of the present disclosure.

FIG. 4 illustrates an example transformation that can be generated using the example method of FIG. 3, in accordance with embodiments of the present disclosure.

FIG. 5 illustrates a flowchart of an example method for generating artificial test data in the example method of FIG. 1, in accordance with embodiments of the present disclosure.

FIG. 6 illustrates an example distribution that can be considered in the example method of FIG. 5, in accordance with embodiments of the present disclosure.

FIG. 7 illustrates a high-level block diagram of an example computer system that may be used in implementing one or more of the methods, tools, and modules, and any related functions, described herein, in accordance with embodiments of the present disclosure.

FIG. 8 depicts a cloud computing environment, in accordance with embodiments of the present disclosure.

FIG. 9 depicts abstraction model layers, in accordance with embodiments of the present disclosure.

While the embodiments described herein are amenable to various modifications and alternative forms, specifics thereof have been shown by way of example in the drawings and will be described in detail. It should be understood, however, that the particular embodiments described are not to be taken in a limiting sense. On the contrary, the intention is to cover all modifications, equivalents, and alternatives falling within the spirit and scope of the invention.

### DETAILED DESCRIPTION

Aspects of the present disclosure relate generally to the field of computer aided diagnosis (CAD) systems, and more particularly to the use of CAD systems in clinical modeling. While the present disclosure is not necessarily limited to such applications, various aspects of the disclosure may be appreciated through a discussion of various examples using this context.

Clinical models are artificial intelligence (AI) models that are generated by machine learning algorithms based on input datasets. As used herein, the term “dataset” refers to a set of data samples. Machine learning commonly utilizes three different types of datasets. Training datasets are sets of sample data that are used to train the model. Validation datasets are sets of sample data that are used to compare performances of different trained models and determine which is more appropriate. Finally, test datasets are used to assess the performance of the model based on characteristics such as accuracy, specificity, and sensitivity. Typically, an initially provided dataset is divided into a training dataset, a validation dataset, and a test dataset such that each of the training, validation, and test datasets has approximately the same statistical probability distributions. For example, 80% of the data samples from the initial dataset may be dedicated to the training dataset, 10% of the data samples from the initial dataset may be dedicated to the validation dataset, and 10% of the data samples from the initial dataset may be dedicated to the test dataset. In this way, the initial dataset can be used to train, validate, and test the model. This can be advantageous because using data from the same dataset to train, validate, and test the model can prevent new variables from being introduced at different stages of model development by bringing in data from a different dataset.

However, to improve a model's accuracy and reliability it can be helpful to provide the model with training datasets that are robust, that include as much data as possible, and that include sample data that matches as closely as possible real world examples that the model is likely to encounter. This can be difficult to achieve while also using data from a single dataset to train, validate, and test the model.

In particular, clinical models, generated by CAD systems, are built using datasets of sample medical data and are utilized in clinical medical applications to help inform medical professionals in diagnosis and decision making. However, the variety of data and how well the data matches real world examples is dependent on the source of the data. For example, a medical provider in the suburbs surrounding San Francisco will have patients, and therefore be collecting patient data, from different demographics than a medical provider in downtown Jackson, Mississippi. If the sample medical data comes from one of these sites, it may not be representative or translatable to the other site due to differences in age, race, underlying medical conditions, medications being taken, or a number of other factors in the site's patient population that can have a statistically relevant effect on a medical diagnosis, prognosis, treatment, and outcome.

Deep learning is one type of machine learning which utilizes architectures such as artificial neural networks to improve algorithms automatically through experience. Recently, deep learning has been adopted for the development of CAD algorithms in various medical imaging fields. One problem with deep learning algorithms is that the algorithms can be overtrained when there are not enough training datasets or not enough variation in the training datasets that are used to build the model. In overtraining, the algorithm may overcomplicate the training by assigning each data sample its own category, and at the same time, the algorithm may oversimplify the training because it does not have to learn the details of why data samples get categorized the way that they do. The resulting models are inaccurate and cannot be reliably applied to further data samples.

When deep learning is used with medical imaging, where algorithms are trained on images from sites, such as labs or hospitals where testing is performed, overtraining may be inevitable. For example, in circumstances where algorithms are trained on data from a small number of sites, the data is inherently limited by the conditions of the sites. Additionally, in circumstances where algorithms are trained on data for unusual medical conditions, the data is inherently limited by the number of available samples. Accordingly, the algorithms will have gaps in their training data, and the resulting models are unable to be generalized to be deployed at new sites. While some gaps can be addressed by generating or adding more data samples to the training data, with deep learning algorithms, it may be difficult to determine what kind of additional data samples are needed to improve the performance of the resulting model.

Many factors influence the performance of AI-based models in real clinical environments. For example, clinical models are used to evaluate medical images of breast tissue to aid in the diagnosis of breast cancer based on breast tissue density. Breast tissue density varies on an individual basis and is also influenced by factors such as age and race. Accordingly, datasets with medical images taken from sites that have populations with high proportions of a particular age or race can lead to overtraining. Moreover, medical images of breast tissue vary depending on the type or manufacturer of the mammography machine used to produce the image. Accordingly, datasets with medical images taken from sites that have mammography machines from only one manufacturer or a disproportionate representation of manufacturers can also lead to overtraining.

Given the significance and sensitivity of generating accurate and reliable information for medical diagnoses, ensuring that AI-based models are accurate and reliable in a clinical environment is extremely important. However, it is also an expensive and time consuming task. Furthermore, it is also a costly process to deploy a model built using incomplete datasets and then repeatedly discover errors in clinical settings and have to retrain the model with updated and/or improved findings or datasets.

Additionally, clinical models rely on annotations or labels that indicate the diagnosis or outcome associated with a sample image. Currently, there is no systematic way to add these annotations, which may provide crucial data to the algorithm training.

Embodiments of the present disclosure may overcome the above, and other, problems by providing a system that supports the generalization of AI models in real clinical environments. As discussed in further detail below, in at least some embodiments of the present disclosure, the system identifies gaps or other deficiencies in AI training. In at least some embodiments of the present disclosure, the system automatically corrects such issues in the AI training. In at least some embodiments of the present disclosure, the system analyzes limitations of an AI model and adjusts data distribution to correct skewed or disproportionate datasets. In at least some embodiments of the present disclosure, the system applies a restricted generative adversarial network (GAN), discussed in further detail below, to generate test datasets having specific statistical distributions. In at least some embodiments of the present disclosure, the system generates balanced annotation candidates to improve the AI model.

It is to be understood that the aforementioned advantages are example advantages and should not be construed as limiting. Embodiments of the present disclosure can contain all, some, or none of the aforementioned advantages while remaining within the spirit and scope of the present disclosure.

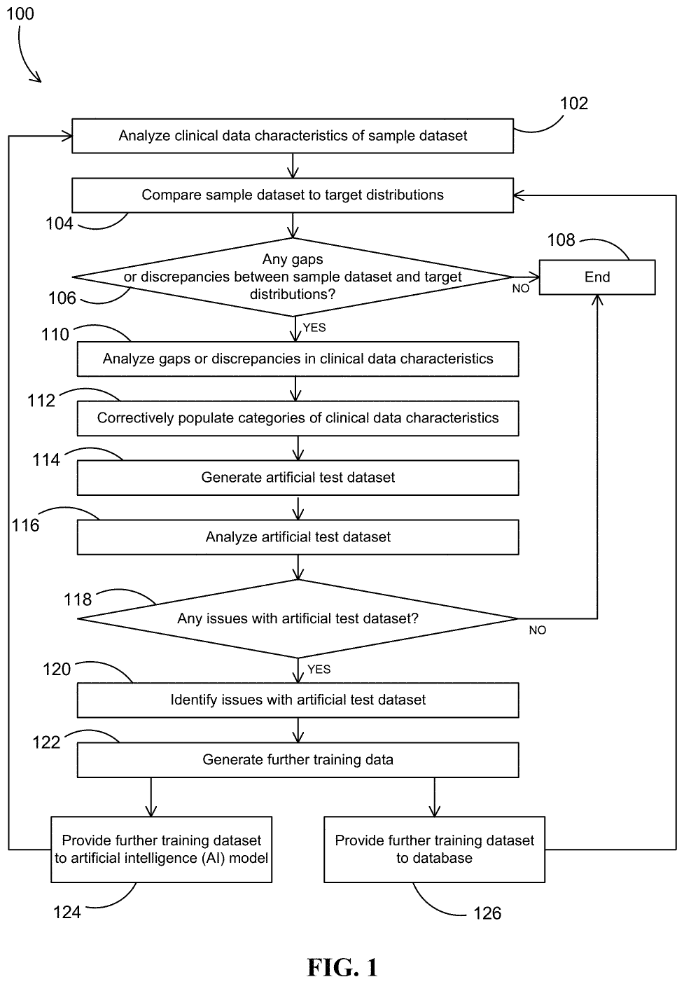

Turning now to the figures, FIG. 1 illustrates a flowchart of an example method 100 for generalizing AI models, in accordance with embodiments of the present disclosure. In an illustrative example used throughout this application, the AI model is applied to a body of sample data including medical images of breast tissue generated by mammography to aid in screening and diagnosis regarding breast cancer. However, it is to be understood that this is an example application of various embodiments disclosed herein provided for illustrative purposes, that the embodiments disclosed herein may be applied to other type of models and/or medical imaging, and that the present disclosure is not limited to analysis of mammography images. Provided with a body of sample data, the method 100 can be used to generalize the model such that the model can be accurately and reliably applied to new or future medical images.

At operation 102, the system analyzes clinical data characteristics relevant to a given AI model. In particular, in order to analyze the clinical data characteristics that are relevant to a given AI model, the system must be provided with an initial sample dataset which includes the relevant clinical data characteristics. The initial sample dataset is a set of genuine or authentic data provided to the system to facilitate training, validation, and testing of the AI model. In at least some embodiments of the present disclosure, analyzing clinical data characteristics includes generating statistical distributions of the clinical data characteristics of the sample dataset.

In the illustrative example, clinical data characteristics can include, without limitation, patient-specific breast tissue density, patient age, patient race, and the manufacturer of the mammography machine used to generate the patient images. Within each characteristic, a number of categories is defined. For example, the clinical data characteristic patient-specific breast tissue density includes density A, density B, density C, and density D.

In the illustrative example, at operation 102, the system analyzes a sample dataset including data provided from North, South, and East sites and generates statistical distributions of the patient-specific breast tissue density, patient age, patient race, and mammography machine manufacturer in the provided sample data.

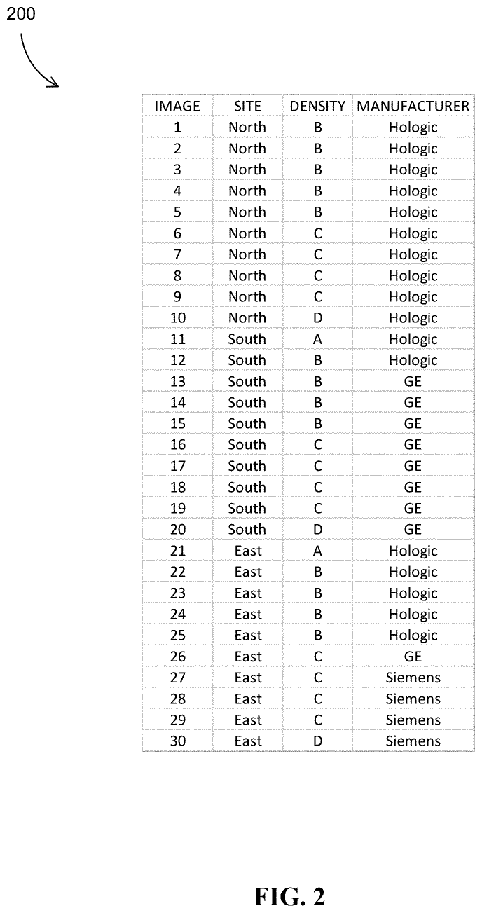

Example data is provided in the table 200 shown in FIG. 2. For the purposes of this illustration, the example data associated with each image only includes the location site, the patient-specific breast tissue density, and the mammography machine manufacturer. However, as mentioned above, additional data associated with each image can include, without limitation, patient age and patient race. Additionally, the example data provided in table 200 includes data associated with ten images from each site. However, example data can include data associated with more or fewer images from more or fewer sites. For the purposes of this illustration, it is assumed that the sample dataset includes more data than is shown in the table 200, and that the example data provided in table 200 is statistically representative of the larger dataset. It is also assumed that the example data provided in table 200 is representative of the entire sample dataset that is subsequently divided into and dedicated to training, validation, and test datasets.

With reference to FIG. 2, analysis of the clinical data characteristics of images provided from the North site generates breast tissue density distributions of 50% density B, 40% density C, and 10% density D and manufacturer distributions of 100% Hologic®. Analysis of the clinical data characteristics of images provided from the South site generates breast tissue density distributions of 10% density A, 40% density B, 40% density C, and 10% density D and manufacturer distributions of 20% Hologic® and 80% General Electric® (GE®). Analysis of the clinical data characteristics of images provided from the East site generates breast tissue density distributions of 10% density A, 40% density B, 40% density C, and 10% density D and manufacturer distributions of 50% Hologic®, 10% GE®, and 40% Siemens®.

When building the model using the provided sample dataset, knowing how the data distributions compare to distributions of clinical data characteristics that occur in the real world, also referred to as target distributions, enable generalization of the model and facilitate an understanding of how accurate and reliable the model can be when applied to new or future data. In other words, in order to generate a model that can be most accurately applied in the real world, the target distributions should be represented as closely as possible in the dataset used to train, validate, and test the AI model.

Accordingly, at operation 104, the system compares the statistical distributions of clinical data characteristics of the sample datasets with the target distributions of the clinical data characteristics. In order to do so, the system must have the statistical distributions of the sample dataset generated by operation 102 as well as target distributions of the same clinical data characteristics.

In the illustrative example, the system must be provided with target distributions of patient-specific breast tissue density and mammography machine manufacturer. Regarding the target distribution of patient-specific breast tissue density, the breast tissue density of approximately 10% of US women is clinically categorized as almost entirely fatty (referred to as “density A”), the breast tissue density of approximately 40% of US women is clinically categorized as scattered areas of fibroglandular density (referred to as “density B”), the breast tissue density of approximately 40% of US women is clinically categorized as heterogeneously dense (referred to as “density C”), and the breast tissue density of approximately 10% of US women is clinically categorized as extremely dense (referred to as “density D”). Accordingly, the target distribution of patient-specific breast tissue density includes a number of data samples per density categorization that is directly proportionate to this statistical distribution.

It should be noted that breast tissue density varies naturally from patient to patient. However, because mammograms rely on the identification and assessment of masses, which appear in mammogram images as areas of higher density in breast tissue, underlying density is an important factor to take into consideration when interpreting mammograms. For example, breast tissue that is extremely dense lowers the sensitivity of the mammography. Additionally, breast tissue that is heterogeneously dense may obscure small masses. It should also be noted that the categorization of breast tissue density is a subjective determination made by the particular radiologist interpreting a particular mammogram.

Regarding the target distribution of mammography machine manufacturer, it is assumed that in the United States, approximately 50% of breast tissue images are produced using mammography machines manufactured by Hologic®, approximately 30% of breast tissue images are produced using mammography machines manufactured by GE®, approximately 10% of breast tissue images are produced using mammography machines manufactured by Siemens®, and approximately 10% of breast tissue images are produced using mammography machines manufactured by Philips®. Accordingly, the target distribution of mammography machine manufacturer includes a number of data samples per manufacturer that is directly proportionate to this statistical distribution.

In the illustrated example, at operation 104, the distributions of patient-specific breast tissue density and mammography machine manufacturer that are generated from the sample dataset in operation 102 are compared with these target distributions.

At operation 106, the system determines from the comparison whether there are any gaps or discrepancies between the statistical distributions of the sample dataset and the target distributions. A gap or discrepancy may exist if the difference between the sample dataset and the target distribution exceeds a threshold. If there are no gaps or discrepancies, then this indicates that the provided sample dataset is sufficient to use to train the clinical model. Thus, in this case, the method proceeds to operation 108, wherein the method ends. Otherwise, if the system determines that there are gaps or discrepancies, then this indicates that the provided sample dataset is not sufficient to use to train the clinical model, but will instead produce an inaccurate or unreliable clinical model. In this case, the method proceeds to operation 110, wherein the system analyzes any gaps or discrepancies between the two distributions in one or more of the clinical data characteristics.

In the illustrative example, at operation 104, the system compares the statistical distributions of the sample dataset with the target distributions and identifies, at operation 106, that the sample data from the North site underrepresents density A (0% of North site images have density A compared to 10% of the target distribution) and overrepresents density B (50% of North site images have density B compared to 40% of the target distribution). Additionally, the system identifies that the sample data from the North site overrepresents Hologic® (100% of North site images are Hologic® compared to 50% of the target distribution) and underrepresents GE®, Siemens®, and Philips® (0% of North site images are GE®, Siemens®, and Philips® compared to 30%, 10% and 10%, respectively).

Similarly, after comparing the statistical distributions of the sample dataset with the target distributions at operation 104, the system identifies, at operation 106, that the sample data from the South site underrepresents Hologic®, Siemens®, and Philips® (20%, 0% and 0% of South site images are Hologic®, Siemens®, and Philips®, respectively, compared to 50%, 10% and 10%) and overrepresents GE® (80% of South site images are GE® compared to 30% of the target distribution).

Similarly, after comparing the statistical distributions of the sample dataset with the target distributions at operation 104, the system identifies, at operation 106, that the sample data from the East site overrepresents Siemens® (40% of East site images are Siemens® compared to 10% of the target distribution) and underrepresents Philips® (0% of East site images are Philips® compared to 10% of the target distribution).

Moreover, in at least some embodiments of the present disclosure, the system also compares the combined statistical distributions of the sample data provided from all three sites relative to the target distributions. In the present example, this aspect of the comparison identifies that the sample dataset completely lacks images that are produced by mammogram machines manufactured by Philips®.

The overrepresentations and underrepresentations discussed above are examples of discrepancies between the distributions of the clinical data characteristics in the sample dataset and the target distributions. The complete lack of images from any site that are produced by mammogram machines manufactured by Philips® is an example of a gap in the data from the sample datasets. Accordingly, in the illustrative example, the method proceeds to operation 110, wherein these gaps and discrepancies are analyzed.

The analysis of the gaps and discrepancies that is performed at operation 110 can identify how the distributions of the sample dataset can be brought more nearly into proportion with the target distributions by adding additional data. At operation 112, the system correctively populates a category of clinical data characteristics in which a gap or discrepancy is discovered by adding additional data to the originally provided datasets. In at least some embodiments of the present disclosure, corrective data can be added by collecting and inputting additional genuine data having the desired clinical data characteristics from additional sites. In at least some embodiments of the present disclosure, the additional genuine data can be collected and input using an online learning-based system. In at least some embodiments of the present disclosure, the additional genuine data can be manually collected and input. In at least some alternative embodiments of the present disclosure, at operation 112, the system correctively populates a category of clinical data characteristics in which a gap or discrepancy is discovered by filtering out or removing some of the existing data.

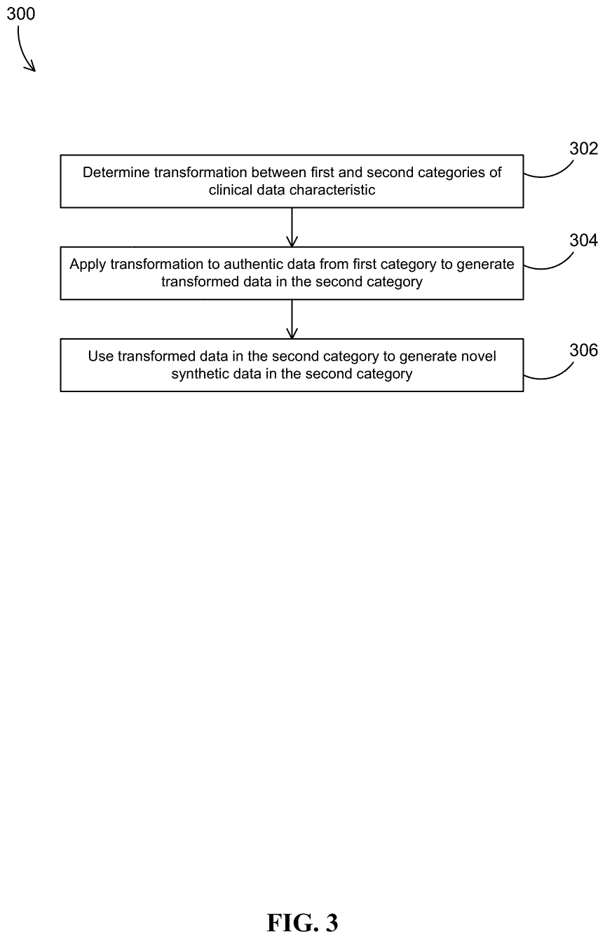

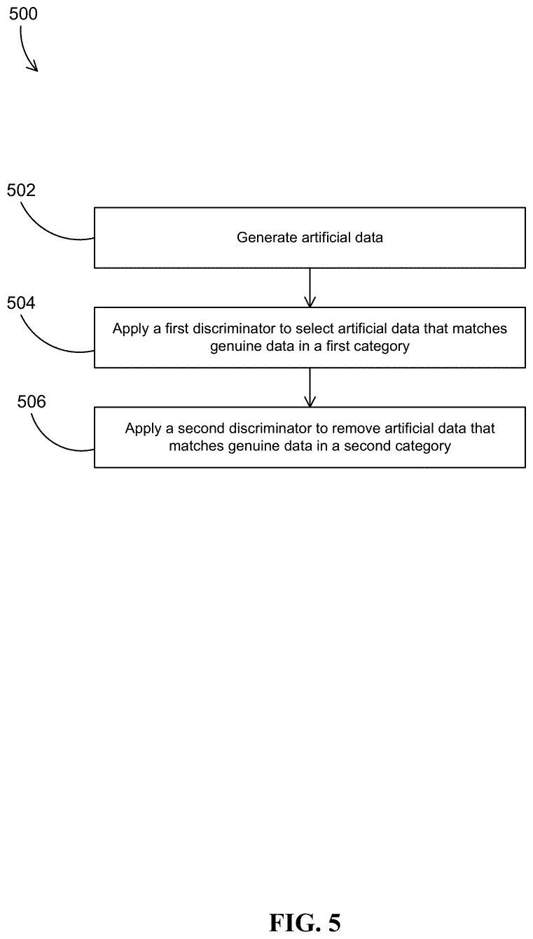

At operation 114, the system generates an artificial or synthetic test dataset based on the target distributions. In at least some embodiments of the present disclosure, the synthetic test dataset includes distribution-specific images. In such embodiments, the set of images that are generated to make up the synthetic test dataset has clinical data characteristics based on the target distributions and the gaps or discrepancies between the distributions of existing dataset and the target distributions. How the system generates the synthetic test dataset is described in further detail below with reference to methods 300 and 500, shown in FIGS. 3 and 5, respectively.

In the illustrative example, the system generates images to address the underrepresentation of density A from the North site, the overrepresentation of density B from the North site, the overrepresentation of Hologic® from the North site, the underrepresentation of GE®, Siemens®, and Philips® from the North site, the underrepresentation of Hologic®, Siemens®, and Philips® from the South site, the overrepresentation of GE® from the South site, the overrepresentation of Siemens® from the East site, and the underrepresentation of Philips® from the East site. The system also generates images to correct for the gap in Philips® images from the North, South, and East sites.

More specifically, the system generates such images while also taking into consideration the other clinical data characteristics. For example, when generating images at operation 114, the system corrects for the number of GE® images from the South site while also considering the density distribution of the set of images that it is generating. In other words, because each image will be categorized across multiple clinical data characteristics, the system takes this into account by balancing combinations of clinical data characteristics, not just each clinical data characteristic individually.

Similarly, for example, the system considers the disease distribution when generating Siemens® images for the North site at operation 114. If the system were to generate a disproportionate number of Siemens® images for the North site that were categorized or annotated as including potentially cancerous masses, this influx in the disease distribution would skew the dataset and reduce the accuracy and reliability of the resulting model.

At operation 116, the system performs a statistical analysis of the performance of the model using a test dataset. In at least some embodiments of the present disclosure, the test dataset includes the synthetic data generated at operation 114. In at least some embodiments of the present disclosure, the test dataset includes a combination of the genuine and synthetic data. In at least some embodiments of the present disclosure, the system can perform this statistical analysis of multiple different test datasets to determine which test dataset produces the most accurate and reliable outcomes. This analysis can be used to determine whether or not the dataset needs further adjustment to adequately train the model.

At operation 118, the system determines from the analysis whether there are any issues with the test dataset. The system may perform an image quality check on the generated images in the test dataset to determine whether there are any issues with the test dataset. The image quality check may involve comparing features of the generated images to expected features of those images. The features that are compared may depend on the type of images analyzes. Following the mammogram image analysis example discussed herein, the system can analyze numerous features of the generated images, including, but not limited to, smoothness of tissue boundaries, contrast in the images, intensity distribution, and/or other artifacts. The analysis may include comparing the features to expected values to determine if the features are consistent with (e.g., within a threshold of) what is expected of actual images. Based on the analyzed features of the images, the system can determine whether there are issues with the test dataset.

If there are no issues, then this indicates that the test dataset is sufficient to use to train the clinical model. Thus, in this case, the method proceeds to operation 108, wherein the method ends. Otherwise, if the system determines that there are remaining issues with the test dataset, then this indicates that the test dataset is not sufficient to use to train the clinical model, but will instead produce an inaccurate or unreliable clinical model. In this case, the method proceeds to operation 120, wherein the system identifies any such issues.

In at least some embodiments of the present disclosure, at operation 120, the system identifies issues with the test dataset by generating a performance report. In at least some embodiments of the present disclosure, identifying issues with the test dataset at operation 120 also includes identifying what additional data is still needed to be added to the test dataset to build an accurate and reliable model. In at least some embodiments of the present disclosure, the system suggests an optimal dataset for annotation. In other words, the system suggests an optimized training dataset to be used to train the model. To be most effective, this optimized training data should include explicit annotations or labels that provide the most complete data possible to the model for training.

For example, in the illustrative example, following the performance analysis at operation 116, the system may identify at operation 118 that the model performs poorly when applied to Siemens® images. In other words, the model produces inaccurate or unreliable results when the images it evaluates are Siemens® images. Accordingly, at operation 120, the system generates a performance report identifying this issue and identifying the need to add additional genuine Siemens® images to the test dataset to build an accurate and reliable model.

As another example, the system may identify at operation 118 that the model could be improved by adding additional Philips® images that include annotations. At operation 120, the system generates a performance report identifying this issue and identifying a number of annotated Philips® images required to bring the dataset into conformity with the real world population, indicated by the target distributions.

At operation 122, the system generates further training data to address the specific identified issues. In at least some embodiments of the present disclosure, generating further training data includes generating further synthetic data to address the identified issues. In at least some embodiments of the present disclosure, generating further training data additionally or alternatively includes collecting new genuine data. In at least some embodiments of the present disclosure, generating further training data at operation 122 further includes annotating the further synthetic data and/or new genuine data to assist the model in correctly analyzing the new genuine data during subsequent training.

In the illustrative example, generating further training data includes collecting new genuine data, for example from different sites, that includes Siemens® images. The new genuine data is annotated to assist the model in correctly analyzing the new genuine data during subsequent training.

At operation 124, the system retrains the model using the further training data. In other words, the further training data generated at operation 122 is used to retrain the model to improve the performance of the model. Accordingly, following operation 124, the method 100 returns to operation 102 and begins again. In this way, the system can assess the accuracy and reliability of the model using the updated data generated through the method.

Additionally, at operation 126, the system adds the further training data to a database where all of the data for the system is stored. The further training data has been generated specifically to fill or correct for any gaps or discrepancies in the data. Accordingly, by adding the further training data to the database, the system fills or corrects for the previously identified gaps or discrepancies. Thus, in future iterations of the method 100, the system can then call on this further training data when performing operation 104.

In the embodiment of the method 100 shown in FIG. 1, operations 124 and 126 are both performed following operation 122. In alternative embodiments, however, only one or the other of operations 124 and 126 may be performed. Additionally, operation 124 may be performed before, after, or at approximately the same time as operation 126.

As mentioned above, at operation 114, the system generates an artificial or synthetic test dataset based on the target distribution. Depending on the particular data that is provided and the particular data that is missing, the system generates the synthetic test dataset by performing at least one of a number of methods. One example method 300 for generating a synthetic test dataset is shown in FIG. 3. Another example method 500 for generating a synthetic test dataset is shown in FIG. 5.

More specifically, the method 300 is used to generate one or more synthetic test datasets when the system has determined that the sample dataset has data in a first category of a clinical data characteristic but is missing data in a second category of the clinical data characteristic that is represented in the target distributions. In the illustrative example, the method 300 is used to generate one or more synthetic test datasets because the system has determined that the sample dataset from the North site has images in the Hologic® category of the mammography machine manufacturer but is missing images in the GE® category of the mammography machine manufacturer clinical data characteristic.

At operation 302, the system determines a transformation between the first category and the second category of the clinical data characteristic. More specifically, the system identifies data in the sample dataset from the first category of clinical data characteristics and from the second category of clinical data characteristics and uses that data to determine a transformation that can be applied to data in the first category to generate data in the second category data. To improve the accuracy of the transformation, the identified data from the first category and the identified data from the second category that are used to determine the transformation should have similar statistical distributions in other clinical data characteristics. In other words, the more similar the data samples that are used to generate the transformation, the more accurate the resulting transformation.

In the illustrative example, the system identifies data from the South and East sites in the sample dataset that is associated with images produced by Hologic® mammography machines and images produced by GE® mammography machines. Preferably, the system identifies data from Hologic® images and data from GE® images that have similar density distributions. The system then determines a transformation that can be applied to the Hologic® images to generate synthetic GE® images. Because the system also has genuine GE® images, the system can check the accuracy of its transformation.

In at least some embodiments of the present disclosure, the system uses a cycle GAN to determine the transformation. The cycle GAN uses genuine data from both the first and second categories in order to determine the transformation. More specifically, the cycle GAN uses machine learning to determine how to change superficial characteristics of the data, like the appearance of the image, while keeping the crucial underlying data the same. This is appropriate for the illustrative example because it allows the system to determine a transformation that retains the important anatomical and physiological data of the image, which are used for screening and diagnosis, while transforming only those details of the image that differ between the type of mammography machine that is used to produce the images.

Accordingly, in the illustrative example, the system trains a cycle GAN to find the average transformation from Hologic® images to GE® images between the South and East sites by combining their Hologic® and GE® data into two training sets for the cycle GAN. To generate the transformation, it is not necessary to have Hologic® and GE® data from both the South and East sites. Instead, the transformation can be determined from the Hologic® and GE® data from either site alone. However, it can be advantageous to use the data from both sites because each site may have a different patient population and/or may calibrate their mammogram machines differently. Accordingly, using data from multiple sites can help mitigate the impact of peculiarities from any particular site on the training of the cycle GAN and the resulting transformation.

At operation 304, the system applies the transformation to the data from the sample dataset having the first category of clinical data characteristics to generate synthetic data having clinical data characteristics in the second category. In this way, the system can correct for the missing data and generate a more robust training dataset for the model.

In the illustrative example, the system applies the transformation determined using the Hologic® and GE® images from the South and East sites to the Hologic® images from the North site to generate synthetic GE® images from the North site.

At operation 306, the system uses the generated synthetic data having clinical data characteristics in the second category to generate novel synthetic data having clinical data characteristics in the second category. In other words, the novel synthetic data having clinical data characteristics in the second category is not a transformation of genuine data having clinical data characteristics in the first category. Instead, the novel synthetic data is new training data, which has distributions of other clinical data characteristics that match target distributions. Otherwise, the synthetic data would merely duplicate the other clinical data characteristics that were already represented in the genuine data, skewing the training data. In at least some embodiments of the present disclosure, the system can generate the novel synthetic data using a progressive GAN.

In the illustrative example, the system uses the generated synthetic GE® images from the North site to generate novel synthetic GE® images having other clinical data characteristic distributions based on the target distributions. For example, the system may generate novel synthetic images from the North site having density distributions that match the target density distributions. By providing a training dataset with clinical data characteristic distributions that more closely matches the target distributions, the system is able to generate a more accurate and reliable model that can be generalized to new or future datasets.

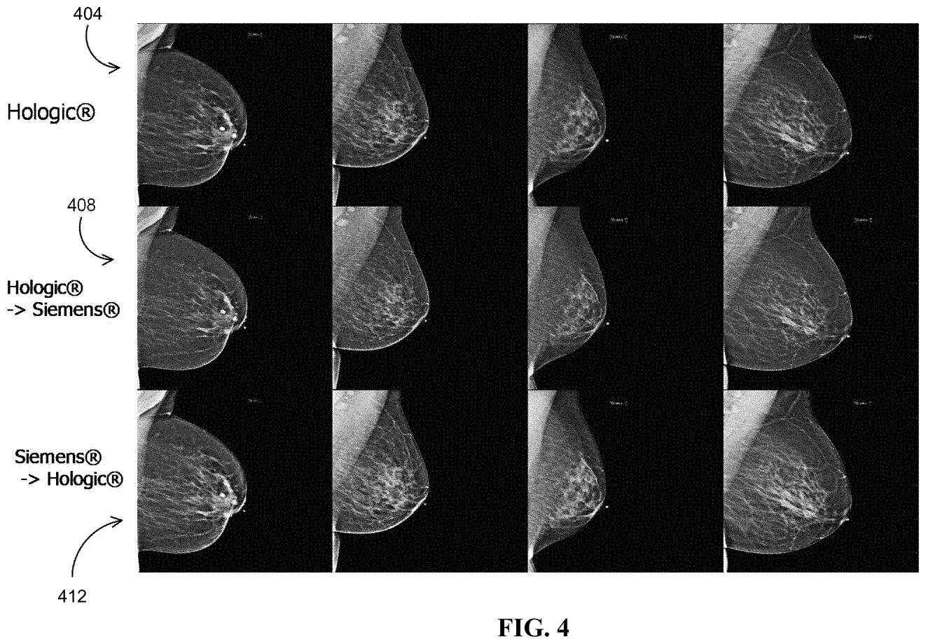

In another application of the illustrative example, as shown in FIG. 4, the system can perform the method 300 to train a cycle GAN to find a transformation from Hologic® images to Siemens® images using the Hologic® and Siemens® data from the East site. By performing the method 300, the system determines a transformation that can be applied to the initial images from Hologic®, shown in row 404, to generate images that are reliably and accurately identified and interpreted as images from Siemens®, shown in row 408. Once the transformation has been determined, the system can generate a novel synthetic test dataset that includes synthetic Siemens® images, shown in row 408.

As further illustrated in FIG. 4, in at least some embodiments of the present disclosure, to verify the reliability and accuracy of the transformation, the system also reverses the transformation to transform the Siemens® images, shown in row 408, back into Hologic® images, shown in row 412. The resulting Hologic® images, shown in row 412, can then be compared with the initial Hologic® images, shown in row 404, to ensure that the relevant data contained in the resulting Hologic® images, shown in row 412, is identical to that of the initial Hologic® images, shown in row 404. In such embodiments, this reverse transformation and comparison can also verify that no data was lost or compromised by the transformation and reverse transformation of the images.

Turning now to FIG. 5, the method 500 is used to generate one or more synthetic test datasets when the system has determined that one category of a clinical data characteristic is underrepresented relative to the target distribution of that clinical data characteristic. In the illustrative example, the method 500 can be used to generate images having density A to correct for an underrepresentation of density A images in the sample dataset.

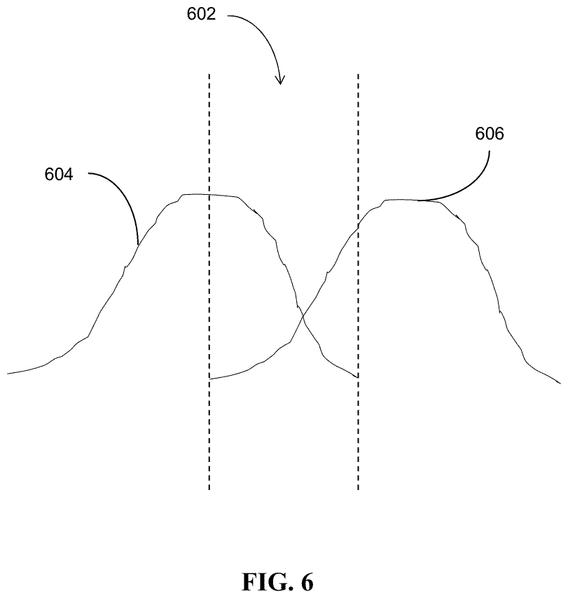

As discussed above, in the illustrative example, whether images are labeled as having density A or density B is a subjective determination. Accordingly, as shown in FIG. 6, there is some overlap 602 in the categorization of images identified as having density A, shown in distribution curve 604, and images identified as having density B, shown in distribution curve 606. In other words, some images could reasonably be categorized as having either density A or density B. In order to address the issue of having too few images categorized as having density A, it is desirable to avoid generating synthetic images that fall into this overlap 602. Generating images that fall into the overlap and then using those images to train the model could exacerbate the problem of having too few images identified as having density A.

At operation 502, the system generates synthetic images (e.g., artificial data) in the underrepresented category of the clinical data characteristic. More specifically, the system inputs a specific subset of the sample dataset into a generator of a multi-discriminator or restricted GAN. The subset of the sample dataset are all images in the underrepresented category of the clinical data characteristic. The generator then generates synthetic images in the same category based on these genuine images.

In the illustrative example, the system inputs images identified as density A, which is underrepresented in the sample dataset relative to the target distribution, into the generator of the restricted GAN to generate synthetic images having density A.

At operation 504, the system applies a first discriminator to the synthetic images generated at operation 502. The first discriminator selects those images that are identified as being in the underrepresented category. In other words, the first discriminator checks to verify that all of the images generated at operation 502 are, in fact, identified as being in the underrepresented category of the clinical data characteristic. In at least some embodiments of the present disclosure, any images that do not get identified as being in the underrepresented category can be filtered out of or removed from the set of synthetic images.

In the illustrative example, the system applies a first discriminator to the synthetic images having density A to verify that all of the synthetic images do, in fact, get classified as density A images. In at least some embodiments of the present disclosure, any images that do not get classified as density A images can be filtered out of or removed from the set of synthetic images.

At operation 506, the system applies a second discriminator to the synthetic images. In embodiments where images that did not get identified as being in the underrepresented category were removed from the set of synthetic images, the system applies the second discriminator to the remaining synthetic images. The second discriminator checks that none of the synthetic images (or remaining synthetic images) are in a second category that overlaps with the underrepresented category of the clinical data characteristic. In order to apply the second discriminator, the system must also be supplied with a subset of the sample dataset that are images in this second, overlapping category in order to be trained to recognize images that will be identified as falling within the second category. In at least some embodiments of the present disclosure, any images that do get classified has being in the overlapping category can be filtered out of or removed from the set of synthetic images.

In the illustrative example, the system applies the second discriminator to the remaining synthetic images identified as having density A to verify that none of the images get classified as having density B. In this way, the system eliminates those synthetic images that fall into the overlap. Thus, the system generates only images having density A, to address the underrepresentation of density A images in the sample dataset, without incidentally also generating images that could also be classified as having density B, which would be counterproductive.

In at least some alternative embodiments of the present disclosure, the same method 500 can be performed using different data subsets and different discriminators to generate specific images that avoid falling into other overlapping categories as well.

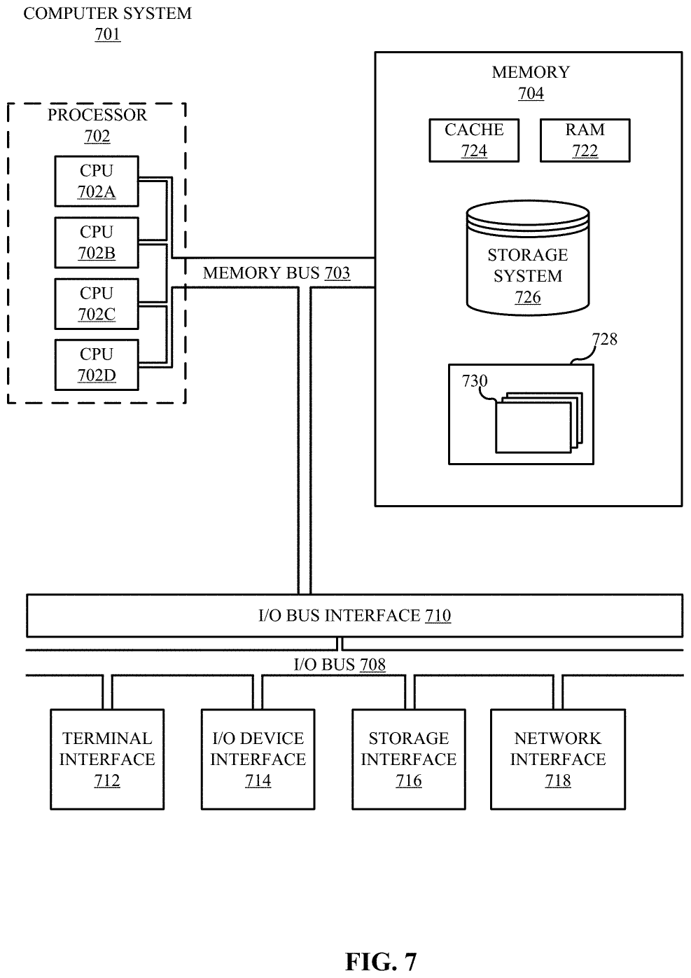

Referring now to FIG. 7, shown is a high-level block diagram of an example computer system 701 that may be used in implementing one or more of the methods, tools, and modules, and any related functions, described herein (e.g., using one or more processor circuits or computer processors of the computer), in accordance with embodiments of the present disclosure. In some embodiments, the major components of the computer system 701 may comprise one or more CPUs 702, a memory subsystem 704, a terminal interface 712, a storage interface 716, an I/O (Input/Output) device interface 714, and a network interface 718, all of which may be communicatively coupled, directly or indirectly, for inter-component communication via a memory bus 703, an I/O bus 708, and an I/O bus interface unit 710.

The computer system 701 may contain one or more general-purpose programmable central processing units (CPUs) 702A, 702B, 702C, and 702D, herein generically referred to as the CPU 702. In some embodiments, the computer system 701 may contain multiple processors typical of a relatively large system; however, in other embodiments the computer system 701 may alternatively be a single CPU system. Each CPU 702 may execute instructions stored in the memory subsystem 704 and may include one or more levels of on-board cache.

System memory 704 may include computer system readable media in the form of volatile memory, such as random access memory (RAM) 722 or cache memory 724. Computer system 701 may further include other removable/non-removable, volatile/non-volatile computer system storage media. By way of example only, storage system 726 can be provided for reading from and writing to a non-removable, non-volatile magnetic media, such as a “hard drive.” Although not shown, a magnetic disk drive for reading from and writing to a removable, non-volatile magnetic disk (e.g., a “floppy disk”), or an optical disk drive for reading from or writing to a removable, non-volatile optical disc such as a CD-ROM, DVD-ROM or other optical media can be provided. In addition, memory 704 can include flash memory, e.g., a flash memory stick drive or a flash drive. Memory devices can be connected to memory bus 703 by one or more data media interfaces. The memory 704 may include at least one program product having a set (e.g., at least one) of program modules that are configured to carry out the functions of various embodiments.

One or more programs/utilities 728, each having at least one set of program modules 730 may be stored in memory 704. The programs/utilities 728 may include a hypervisor (also referred to as a virtual machine monitor), one or more operating systems, one or more application programs, other program modules, and program data. Each of the operating systems, one or more application programs, other program modules, and program data or some combination thereof, may include an implementation of a networking environment. Program modules 730 generally perform the functions or methodologies of various embodiments.

Although the memory bus 703 is shown in FIG. 7 as a single bus structure providing a direct communication path among the CPUs 702, the memory subsystem 704, and the I/O bus interface 710, the memory bus 703 may, in some embodiments, include multiple different buses or communication paths, which may be arranged in any of various forms, such as point-to-point links in hierarchical, star or web configurations, multiple hierarchical buses, parallel and redundant paths, or any other appropriate type of configuration. Furthermore, while the I/O bus interface 710 and the I/O bus 708 are shown as single respective units, the computer system 701 may, in some embodiments, contain multiple I/O bus interface units 710, multiple I/O buses 708, or both. Further, while multiple I/O interface units are shown, which separate the I/O bus 708 from various communications paths running to the various I/O devices, in other embodiments some or all of the I/O devices may be connected directly to one or more system I/O buses.

In some embodiments, the computer system 701 may be a multi-user mainframe computer system, a single-user system, or a server computer or similar device that has little or no direct user interface, but receives requests from other computer systems (clients). Further, in some embodiments, the computer system 701 may be implemented as a desktop computer, portable computer, laptop or notebook computer, tablet computer, pocket computer, telephone, smart phone, network switches or routers, or any other appropriate type of electronic device.

It is noted that FIG. 7 is intended to depict the representative major components of an exemplary computer system 701. In some embodiments, however, individual components may have greater or lesser complexity than as represented in FIG. 7, components other than or in addition to those shown in FIG. 7 may be present, and the number, type, and configuration of such components may vary.

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

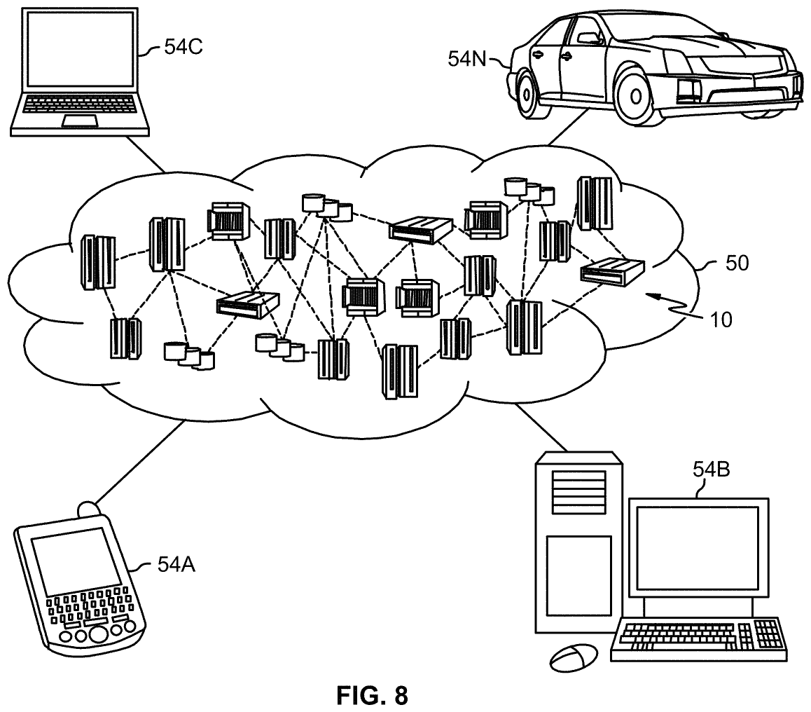

Referring now to FIG. 8, illustrative cloud computing environment 50 is depicted. As shown, cloud computing environment 50 comprises one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 8 are intended to be illustrative only and that computing nodes 10 and cloud computing environment 50 can communicate with any type of computerized device over any type of network and/or network addressable connection (e.g., using a web browser).

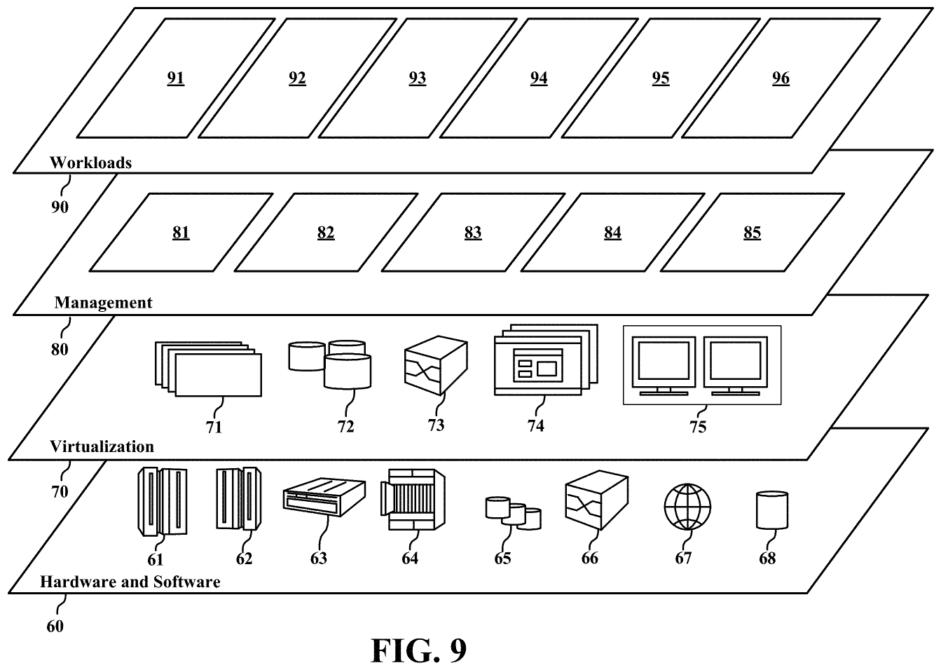

Referring now to FIG. 9, a set of functional abstraction layers provided by cloud computing environment 50 (FIG. 8) is shown. It should be understood in advance that the components, layers, and functions shown in FIG. 9 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and clinical model validation 96.

In addition to embodiments described above, other embodiments having fewer operational steps, more operational steps, or different operational steps are contemplated. Also, some embodiments may perform some or all of the above operational steps in a different order. Furthermore, multiple operations may occur at the same time or as an internal part of a larger process. The modules are listed and described illustratively according to an embodiment and are not meant to indicate necessity of a particular module or exclusivity of other potential modules (or functions/purposes as applied to a specific module).

In the foregoing, reference is made to various embodiments. It should be understood, however, that this disclosure is not limited to the specifically described embodiments. Instead, any combination of the described features and elements, whether related to different embodiments or not, is contemplated to implement and practice this disclosure. Many modifications and variations may be apparent to those of ordinary skill in the art without departing from the scope and spirit of the described embodiments. Furthermore, although embodiments of this disclosure may achieve advantages over other possible solutions or over the prior art, whether or not a particular advantage is achieved by a given embodiment is not limiting of this disclosure. Thus, the described aspects, features, embodiments, and advantages are merely illustrative and are not considered elements or limitations of the appended claims except where explicitly recited in a claim(s).

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the various embodiments. As used herein, the singular forms “a,” “an,” and “the” are intended to include the plural forms as well, unless the context clearly indicates otherwise. It will be further understood that the terms “includes” and/or “including,” when used in this specification, specify the presence of the stated features, integers, steps, operations, elements, and/or components, but do not preclude the presence or addition of one or more other features, integers, steps, operations, elements, components, and/or groups thereof. In the previous detailed description of example embodiments of the various embodiments, reference was made to the accompanying drawings (where like numbers represent like elements), which form a part hereof, and in which is shown by way of illustration specific example embodiments in which the various embodiments may be practiced. These embodiments were described in sufficient detail to enable those skilled in the art to practice the embodiments, but other embodiments may be used and logical, mechanical, electrical, and other changes may be made without departing from the scope of the various embodiments. In the previous description, numerous specific details were set forth to provide a thorough understanding the various embodiments. But, the various embodiments may be practiced without these specific details. In other instances, well-known circuits, structures, and techniques have not been shown in detail in order not to obscure embodiments.

As used herein, “a number of” when used with reference to items, means one or more items. For example, “a number of different types of networks” is one or more different types of networks.

When different reference numbers comprise a common number followed by differing letters (e.g., 100a, 100b, 100c) or punctuation followed by differing numbers (e.g., 100-1, 100-2, or 100.1, 100.2), use of the reference character only without the letter or following numbers (e.g., 100) may refer to the group of elements as a whole, any subset of the group, or an example specimen of the group.

Further, the phrase “at least one of,” when used with a list of items, means different combinations of one or more of the listed items can be used, and only one of each item in the list may be needed. In other words, “at least one of” means any combination of items and number of items may be used from the list, but not all of the items in the list are required. The item can be a particular object, a thing, or a category.

For example, without limitation, “at least one of item A, item B, or item C” may include item A, item A and item B, or item B. This example also may include item A, item B, and item C or item B and item C. Of course, any combinations of these items can be present. In some illustrative examples, “at least one of” can be, for example, without limitation, two of item A; one of item B; and ten of item C; four of item B and seven of item C; or other suitable combinations.

Different instances of the word “embodiment” as used within this specification do not necessarily refer to the same embodiment, but they may. Any data and data structures illustrated or described herein are examples only, and in other embodiments, different amounts of data, types of data, fields, numbers and types of fields, field names, numbers and types of rows, records, entries, or organizations of data may be used. In addition, any data may be combined with logic, so that a separate data structure may not be necessary. The previous detailed description is, therefore, not to be taken in a limiting sense.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Although the present invention has been described in terms of specific embodiments, it is anticipated that alterations and modification thereof will become apparent to the skilled in the art. Therefore, it is intended that the following claims be interpreted as covering all such alterations and modifications as fall within the true spirit and scope of the invention.

What is claimed is:

1. A method for adapting an artificial intelligence (AI) model, the method comprising:
comparing a distribution of a clinical data characteristic of a genuine dataset with a target distribution of the clinical data characteristic to identify any categories of the clinical data characteristic that are underrepresented in the genuine dataset;
generating an artificial test dataset based on the result of the comparison;
generating training data based on the artificial test dataset; and
providing the training data to the AI model to adapt the AI model.

2. The method of claim 1, further comprising:
performing a statistical analysis of the artificial test dataset to identify any problems with the artificial test dataset, wherein:
generating the training data includes generating the training data based on the identified problems.

3. The method of claim 1, wherein generating the artificial test dataset includes:
determining a transformation that, when applied to data in a first category of the clinical data characteristic, transforms data in the first category into data in a second category of the clinical data characteristic, wherein:
the second category of the clinical data characteristic is one of the identified underrepresented categories of the clinical data characteristic in the genuine dataset,
the first category of the clinical data characteristic is another category of the clinical data characteristic that is represented in the genuine dataset, and
the first category is different than the second category.

4. The method of claim 3, wherein:
determining the transformation includes utilizing a generative adversarial network.

5. The method of claim 3, wherein generating the artificial test dataset further includes:
applying the transformation to data in the first category in the genuine dataset to generate transformed artificial data in the second category.

6. The method of claim 5, wherein generating the artificial test dataset further includes:
generating novel artificial data in the second category.

7. The method of claim 1, wherein generating the artificial test dataset includes:
generating artificial data in a first category of the clinical data characteristic, wherein the first category is one of the identified underrepresented categories of the clinical data characteristic in the genuine dataset;
applying a first discriminator to artificial data in the first category to select artificial data that is identified as being in the first category; and
applying a second discriminator to the artificial data in the first category to remove artificial data that is identified as being in a second category of the clinical data characteristic, wherein:
the second category of the clinical data characteristic is another category of the clinical data characteristic that is represented in the genuine dataset, and
the first category is different than the second category.

8. The method of claim 7, wherein:
the first discriminator and the second discriminator are utilized in a generative adversarial network.

9. A computer program product comprising a computer readable storage medium having program instructions embodied therewith, the program instructions executable by processor to cause the processor to perform a method comprising:
comparing a distribution of a clinical data characteristic of a genuine dataset with a target distribution of the clinical data characteristic to identify any categories of the clinical data characteristic that are underrepresented in the genuine dataset;
generating an artificial test dataset based on the result of the comparison;
generating training data based on the artificial test dataset; and
providing the training data to the AI model to adapt the AI model.

10. The computer program product of claim 9, wherein the method further comprises:
performing a statistical analysis of the artificial test dataset to identify any problems with the artificial test dataset, wherein:
generating the training data includes generating the training data based on the identified problems.

11. The computer program product of claim 9, wherein generating the artificial test dataset includes:
determining a transformation that, when applied to data in a first category of the clinical data characteristic, transforms data in the first category into data in a second category of the clinical data characteristic, wherein:
the second category of the clinical data characteristic is one of the identified underrepresented categories of the clinical data characteristic in the genuine dataset,
the first category of the clinical data characteristic is another category of the clinical data characteristic that is represented in the genuine dataset, and
the first category is different than the second category.

12. The computer program product of claim 11, wherein generating the artificial test dataset further includes:
applying the transformation to data in the first category in the genuine dataset to generate transformed artificial data in the second category.

13. The computer program product of claim 12, wherein generating the artificial test dataset further includes:
generating novel artificial data in the second category.

14. The computer program product of claim 9, wherein generating the artificial test dataset includes:
generating artificial data in a first category of the clinical data characteristic, wherein the first category is one of the identified underrepresented categories of the clinical data characteristic in the genuine dataset;
applying a first discriminator to the artificial data in the first category to select artificial data that is identified as being in the first category; and
applying a second discriminator to the artificial data in the first category to remove artificial data that is identified as being in a second category of the clinical data characteristic, wherein:
the second category of the clinical data characteristic is another category of the clinical data characteristic that is represented in the genuine dataset, and
the first category is different than the second category.

15. A system configured to adapt an artificial intelligence (AI) model, the system comprising:
a memory; and
a processor communicatively coupled to the memory, wherein the processor is configured to perform a method comprising:
comparing a distribution of a clinical data characteristic of a genuine dataset with a target distribution of the clinical data characteristic to identify any categories of the clinical data characteristic that are underrepresented in the genuine dataset;
generating an artificial test dataset based on the result of the comparison;
generating training data based on the artificial test dataset; and
providing the training data to the AI model to adapt the AI model.

16. The system of claim 15, wherein the method further comprises:
performing a statistical analysis of the artificial test dataset to identify any problems with the artificial test dataset, wherein:
generating the training data includes generating the training data based on the identified problems.

17. The system of claim 15, wherein generating the artificial test dataset includes:
determining a transformation that, when applied to data in a first category of the clinical data characteristic, transforms data in the first category into data in a second category of the clinical data characteristic, wherein:
the second category of the clinical data characteristic is one of the identified underrepresented categories of the clinical data characteristic in the genuine dataset,
the first category of the clinical data characteristic is another category of the clinical data characteristic that is represented in the genuine dataset, and
the first category is different than the second category.

18. The system of claim 17, wherein generating the artificial test dataset further includes:
applying the transformation to data in the first category in the genuine dataset to generate transformed artificial data in the second category.

19. The system of claim 18, wherein generating the artificial test dataset further includes:
generating novel artificial data in the second category.

20. The system of claim 15, wherein generating the artificial test dataset includes:
generating artificial data in a first category of the clinical data characteristic, wherein the first category is one of the identified underrepresented categories of the clinical data characteristic in the genuine dataset;
applying a first discriminator to the artificial data in the first category to select artificial data that is identified as being in the first category; and
applying a second discriminator to the artificial data in the first category to remove artificial data that is identified as being in a second category of the clinical data characteristic, wherein:
the second category of the clinical data characteristic is another category of the clinical data characteristic that is represented in the genuine dataset, and
the first category is different than the second category.

21. A method for adapting an artificial intelligence (AI) model, the method comprising:
comparing a distribution of a clinical data characteristic of a genuine dataset with a target distribution of the clinical data characteristic to identify an underrepresented category of the clinical data characteristic;
generating artificial data in the underrepresented category in the genuine dataset;
categorizing the artificial data;
applying a first discriminator to the artificial data to select artificial data that is categorized in the underrepresented category;
applying a second discriminator to the artificial data to remove artificial data that is categorized in a second category of the clinical data characteristic, the second category of the clinical data characteristic being another category of the clinical data characteristic that is represented in the genuine dataset, and the underrepresented category being different than the second category;
performing a statistical analysis of the artificial data to identify any problems with the artificial data;
generating training data based on the identified problems; and
providing the training data to the AI model to adapt the AI model.

22. A method for clinical model generalization, comprising:
analyzing at least one clinical data characteristic of a sample dataset;
identifying a category of the at least one clinical data characteristic in which there is a discrepancy between an analyzed statistical distribution of data in the identified category and a target statistical distribution of data in the identified category;
generating synthetic data in the identified category based on the target statistical distribution of data in the identified category;
performing a performance analysis of the synthetic data to identify a problem with the synthetic data; and
generating training data to address the identified problem.

23. The method of claim 22, wherein:
generating synthetic data in the identified category includes generating a plurality of synthetic test datasets; and
performing the performance analysis of the synthetic data further includes performing a performance analysis of each synthetic test dataset of the plurality of synthetic test datasets.

24. The method of claim 22, wherein:
the identified category is missing from the sample dataset,
the sample dataset includes data in a second category of the at least one clinical data characteristic, the second category being different than the identified category,
generating synthetic data in the identified category includes:
using further data in the second category and data in the identified category from a second sample dataset to generate a transformation between data in the second category and the identified category, and
generating novel data in the identified category based on the generated transformation.

25. The method of claim 22, wherein:
the identified category is underrepresented in the sample dataset relative to the target statistical distribution of data in the identified category,
the sample dataset includes data in a second category of the at least one clinical data characteristic, the second category being different than the identified category, and
generating synthetic data in the identified category includes:
generating a synthetic dataset including synthetic data in the identified category, categorizing the synthetic data of the generated synthetic dataset,
selecting the generated synthetic data that is categorized in the identified category, and
removing the generated synthetic data that is categorized in the second category.
